(19) United States
(12) Patent Application Publication
(10) Pub. No.: US 2013/0263858 A1
(43) Pub. Date: Oct. 10, 2013

(54) SUPPORT ELEMENT FOR USE WITH PATIENT INTERFACE DEVICE

(71) Applicants: Peter Chi Fai Ho, Eindhoven (NL); Elizabeth Powell Margaria, Eindhoven (NL)

(72) Inventors: Peter Chi Fai Ho, Eindhoven (NL); Elizabeth Powell Margaria, Eindhoven (NL)

(73) Assignee: KONINKLIJKE PHILIPS N.V., EINDHOVEN (NL)

(21) Appl. No.: 13/994,334

(22) PCT Filed: Dec. 21, 2011
(86) PCT No.: PCT/IB11/55645

§ 371 Date: Jun. 14, 2013

Related U.S. Application Data
(60) Provisional application No. 61/425,440, filed on Dec. 21, 2010.

(51) Int. Cl. A61M 16/06; A61M 16/00

(52) U.S. Cl. CPC A61M 16/0683; A61M 16/0057; A61M 16/0666
USPC 128/205.25; 128/206.21

(57) ABSTRACT

A support element (50) for use with a patient interface system (42) includes a first portion (52) adapted to be coupled to the patient interface system and a second portion (54) having a support face (56) adapted to contact the patient. The first and second portions define a cavity (58) therein. The support face includes an aperture (60) disposed therein that provides access to the cavity.

### CROSS-REFERENCE TO RELATED APPLICATIONS

This patent application claims the priority benefit under 35 U.S. §119(e) of U.S. Provisional Application No. 61/425,440 filed on Dec. 21, 2010, the contents of which are herein incorporated by reference.

### BACKGROUND OF THE INVENTION

1. Field of the Invention

The present invention pertains to devices secured to a human head, such as respiratory masks, and, more particularly, to elements used to help support such devices on a human head.

2. Description of the Related Art

There are numerous situations where it is necessary or desirable to deliver a flow of breathing gas non-invasively to the airway of a patient, i.e., without intubating the patient or surgically inserting a tracheal tube in the esophagus. For example, it is known to ventilate a patient using a technique known as non-invasive ventilation. It is also known to deliver continuous positive airway pressure (CPAP) or variable airway pressure, such as a bi-level pressure that varies with the patient's respiratory cycle or an auto-titrating pressure that varies with the monitored condition of the patient. Typical pressure support therapies are provided to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear assembly having one or more straps adapted to fit over/around the patient's head.

Patients that that require pressure support therapy are often confronted with the problem of finding a suitable patient interface device. In finding a suitable patient interface device, such patients frequently struggle with issues relating to the seal and stability of the patient interface device, the comfort of the patient interface device, the size/weight of the patient interface device, and the sizing of the patient interface device. These challenges, if not addressed properly, can compromise the patient's compliance with the prescribed therapy.

More specifically, during the night, the stability of a mask seal will be challenged by the patient moving about in his or her bed. Changing the head position can lead to air delivery hose torque and general interference with other objects (e.g. pillows, sheets, blankets, etc.). Thus, stability, and consequently seal, is a challenge for any patient interface device in the market.

In addition, patient comfort is an important factor, and can be negatively impacted in many ways. For example, over-tightening of the headgear (to compensate for lack of seal and stability) can increase pressure on the face and head, which in turn can result in pressure points and/or skin breakdown. Other components of the patient interface device (e.g. straps, frames, headgear, etc.) can also add discomfort for the patient because the geometry of such components can conflict with facial structures.

Furthermore, the general weight of the patient interface device can negatively impact a patient's experience by causing additional facial pressure or compounding over-tightening issues. Patient interface device weight can also negatively affect the seal and stability. Certain patients also have problems with a patient interface device interfering with his or her line of sight, and overall patient interface device size has been known to cause claustrophobia in some patients.

Finally, properly sizing a patient interface device can be difficult because facial structures vary greatly among patients. Not all patient interface devices are able to accommodate this range of differences, thus requiring either more sizes/variations or not providing an optimal fit for the majority of the user population.

### SUMMARY OF THE INVENTION

Accordingly, it is an object of the present invention to provide a support element for use in securing a patient interface device to the head of a patient that overcomes the shortcomings of conventional patient interface devices and systems. This object is achieved according to one embodiment of the present invention by providing a support element for use with a patient interface system. The support element includes a first portion adapted to be coupled to the patient interface system and a second portion having a support face adapted to contact the patient. The first and second portions define a cavity therein. The support face includes an aperture disposed therein that provides access to the cavity.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention

### BRIEF DESCRIPTION OF THE DRAWINGS

FIG. 1 is a front perspective view of an exemplary embodiment of a patient interface device according to the principles of the present invention shown schematically connected to a pressure support system;

FIG. 2 is a front perspective view of a patient interface system including the patient interface device of FIG. 1 shown being worn by a patient;

FIG. 3 is a rear perspective view of the patient interface device of FIG. 1;

FIG. 4 is a side perspective view of the patient interface device of FIG. 1;

FIG. 5 is a partial cut away view of the patient interface device of FIG. 4 showing details of the support element;

FIG. 6 is a cross-sectional view of a particular embodiment of a support element according to the principles of the present invention;

FIG. 7 is a cross-sectional view of another particular embodiment of a support element according to the principles of the present invention;

FIG. 8 is a cross-sectional view of a support element according to the principles of the present invention showing examples of relevant dimensions;

FIGS. 9A and 9B are top and side views, respectively, of a support element according to the principles of the present invention having a free-form geometry;

FIG. 10 is a cross-sectional of a particular embodiment of a support element according to the principles of the present invention having multiple openings;

FIGS. 11A, 11B and 11C are elevation, isometric, and cross-sectional views, respectively, of support elements of varying size and geometry according to the principles of the present invention; and

FIGS. 12A, 12B and 12C are elevation, isometric, and cross-sectional views, respectively, of support elements of varying size and similar geometry according to the principles of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). As employed herein, the term “and/or” shall mean both alternatives (i.e., “and”) or just one of the alternatives (i.e., “or”).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. Like numbers refer to like elements throughout.

The present invention is directed to a support element that is used in conjunction with patient interface devices such as, for example, without limitation, a nasal pillow or mask. The support element is configured to provide a comfortable and conformable stability in mounting and attaching the mask to the user. More specifically, the element has a self-enclosure system, much like a ball or mushroom shape, with a hollow center. The patient contacting walls collapse and rebound based on the wall thickness. The support element can be used in other areas where patient comfort and/or stability is of concern such as, for example, without limitation, forehead support, cheek pads, etc.

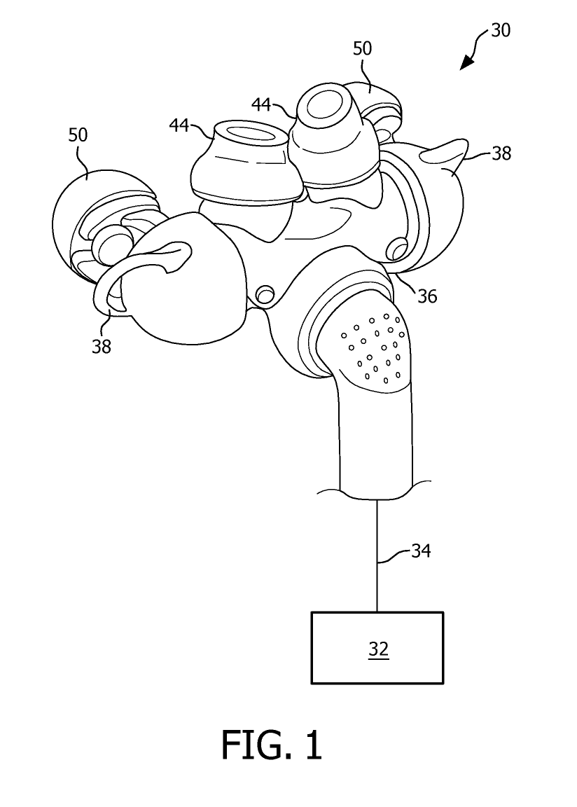

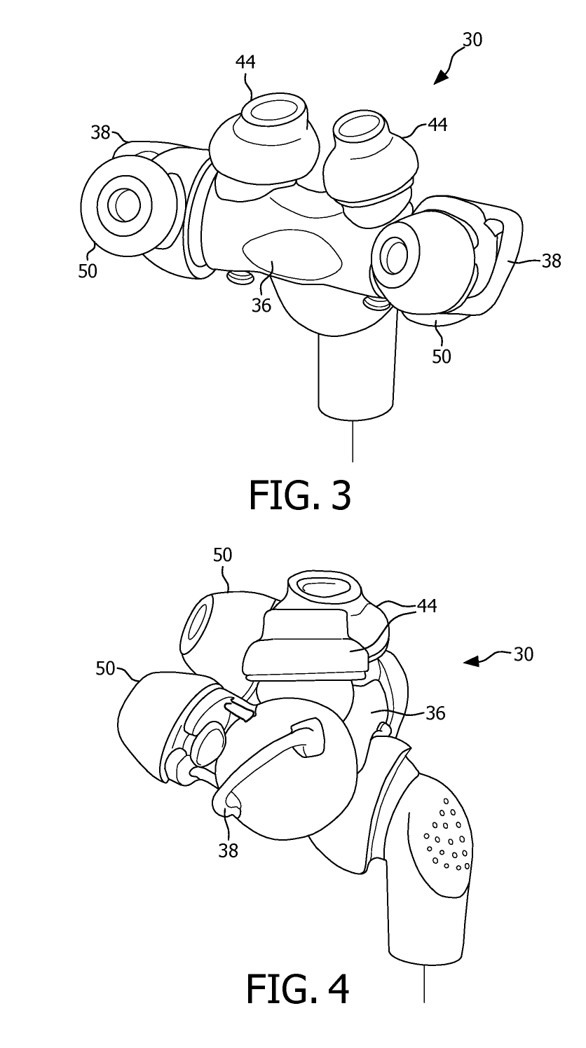

FIGS. 1-5 illustrate an exemplary embodiment of a patient interface device 30 according to the principles of the present invention. Referring to FIG. 1, patient interface device 30 is shown schematically connected to a pressure support system 32 via a patient circuit 34, which communicates gas from the pressure support system to the patient interface device 30. Patient circuit 34 is any device, such as flexible tubing, that carries the flow of gas from the pressure/flow generator in pressure support system 32 to the patient interface device 30. Pressure support system 32 is any conventional ventilation or pressure support system. Examples of such pressure support systems include, but are not limited to: a ventilator, continuous positive airway pressure (CPAP) device, or a variable pressure device, e.g. an auto-titrating device, proportional assist ventilation (PAV®) device, proportional positive airway pressure (PPAP®) device, C-F1ex™ device, Bi-Flex™ device, or a BiPAP® device manufactured and distributed by Respironics, Inc. of Pittsburgh, Pa., in which the pressure provided to the patient varies with the patient's respiratory cycle so that a higher pressure is delivered during inspiration than during expiration, or other pressure support device.

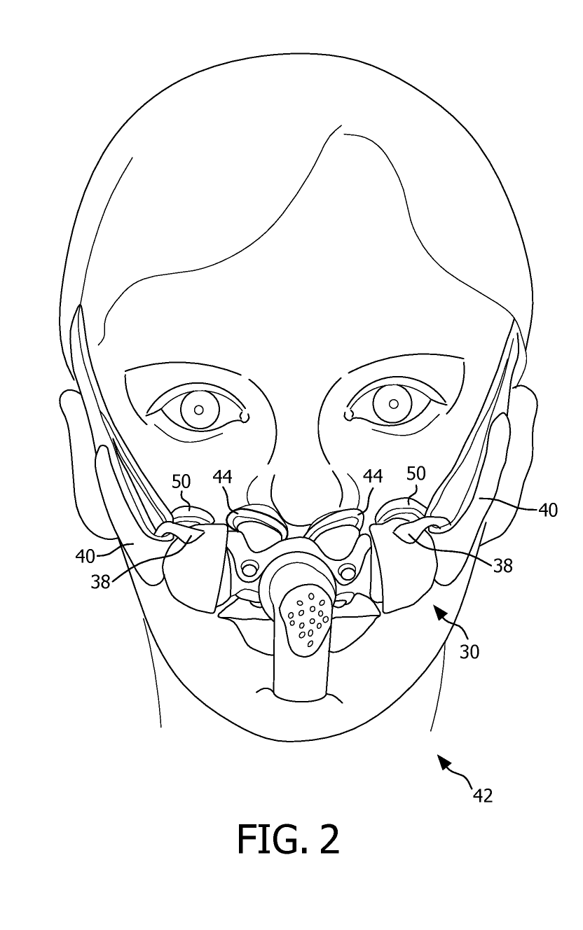

Patient interface device 30 includes a frame 36 sized and configured to span at least a portion of a patient's face while remaining below the patient's eyes when the patient interface device 30 is donned by the patient, as shown in FIG. 2. Frame 36 comprises a generally elongate hollow body and includes headgear attachment elements 38 disposed at each end. In the particular embodiment shown in FIGS. 1-5, each headgear attachment element is in the form of a slot adapted to allow an adjustable strap 40 (FIG. 2) of a headgear assembly (not numbered) to pass therethrough when the patient interface device 30 and headgear assembly are worn by the user as a patient interface system 42 (FIG. 2). In the particular embodiment depicted in FIGS. 1-5, headgear attachment elements 38 are pivotally coupled to frame 36, however, it is to be appreciated that such elements 38 may also be rigidly coupled to frame 36 without varying from the scope of the present invention. Frame 36 and headgear attachment elements 38 are preferably formed from a rigid, lightweight material, such as plastic. Although generally rigid, frame 36 however is preferably slightly bendable to allow flexure when patient interface device 30 is donned by the patient.

Patient interface device 30 further includes a pair of nasal pillows 42 that are positioned to engage and seal against the nares of a patient when patient interface device 30 is secured to the head of a patient, as shown in FIG. 2. A pair of generally deformable support elements 50 are coupled to frame 36 to support the frame on a patient's face. In the exemplary embodiment shown in FIGS. 1-5, support elements 50 are selectively coupled to end portions of the frame 36 adjacent headgear attachment elements 38 such that the support elements overlie the user's zygomatic bones, i.e., cheekbones. This location on the face is believed to be a location that is particularly well suited to support the strapping force imposed on the face when the patient interface device 30 is attached on the head. The size and construction of support elements 50, discussed further below, help disperse the strapping force of the mask over a wide area on the face, thus improving stability and comfort of the mask.

It is to be appreciated that the placement of support elements 50 in FIG. 2 is shown for example purposes only and is not intended to be limiting upon the potential placement of such support elements 50. Accordingly, it is to be further appreciated that such support elements 50 may be positioned in other locations as desired and may accordingly be coupled at other locations on a patient interface assembly, such as, for example, on a portion of the headgear assembly.

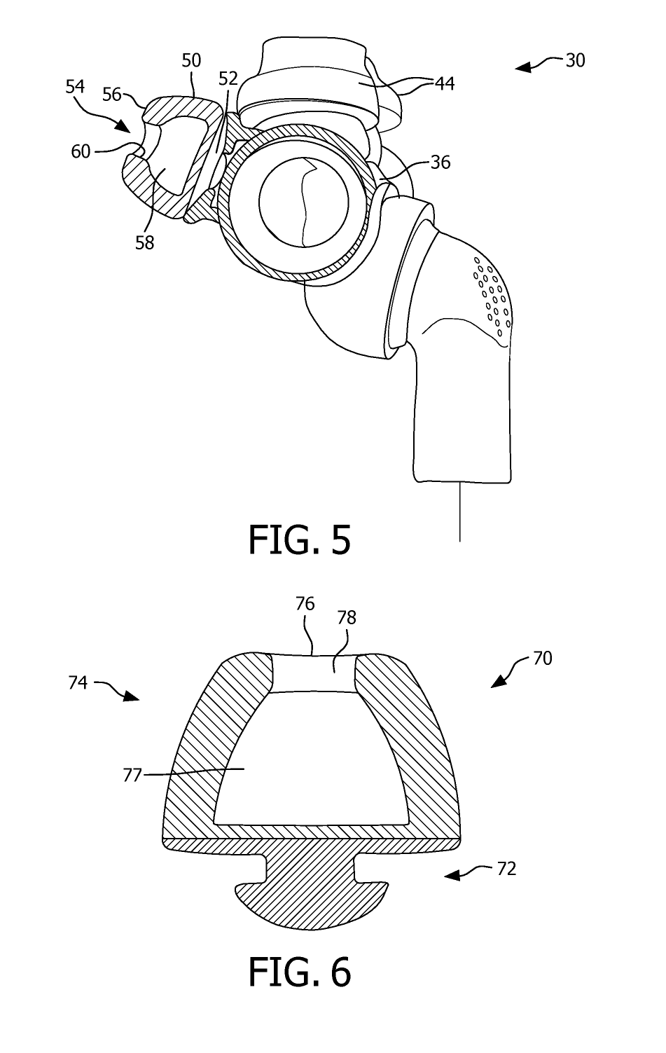

Referring to FIG. 5, support element 50 includes a first portion 52 adapted to be coupled to a portion of the patient interface system and a second portion 54 having a support face 56 that is adapted to contact the patient. In the example shown in FIG. 5, first portion 52 is coupled to a portion of frame 36 via a slide-in button connection mechanism, it is to be appreciated however that first portion 52, and thus support element 50, can be coupled, either permanently or selectively, using any conventional coupling means without varying from the scope of the present invention. Such coupling means may include, for example, without limitation, over-molding and gluing, snap-in or slide-in mechanical locks (similar to that shown) having interlocking elements that can be round, rectangular or in any custom form. Additionally, such coupling may be accomplished using a magnet embedded in the base of the support element that can adhere to a corresponding magnetic area of the patient interface assembly.

Continuing to refer to FIG. 5, first and second portions 52 and 54 of support element 50 are disposed such that a hollow cavity 58 is defined therein. Support face 56 includes an aperture 60 that generally provides access to cavity 58. Such arrangement provides for a support structure having walls 62 that can collapse and rebound in a manner that can be tailored as desired for a specific application depending on the wall thickness and material used.

Having thus described a general overview of a patient interface assembly including a support element 50 according to the principles of the present invention, further examples of support elements that may be used in conjunction with patient interface assemblies with now be described in conjunction with FIGS. 6-10.

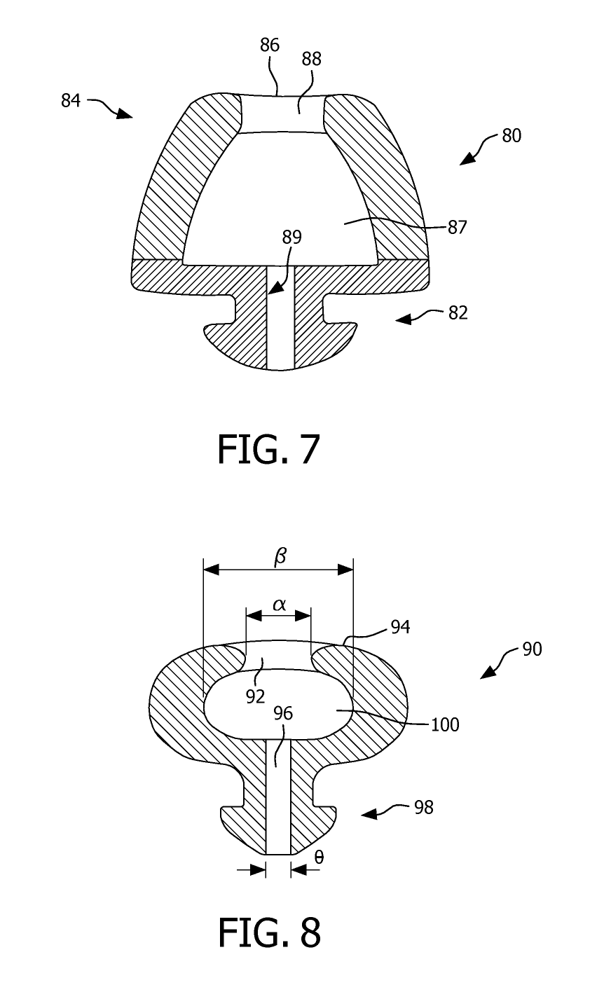

FIGS. 6 and 7 show particular embodiments of support elements 70 and 80 formed from multiple materials. Like support element 50 previously described, support elements 70 and 80 include: first portions 72, 82 adapted to be coupled to a portion of the patient interface system; second portions 74, 84 having a support faces 76, 86 that are adapted to contact the patient; cavities 77, 87 defined therein; and apertures 78,88 formed in support faces 76, 86 that generally provides access to cavities 77 and 87 respectively. Unlike support element 50 which is shown as being formed as a unitary component, first portions 72 and 82 of support elements 70 and 80, respectively, are preferably formed from a generally stiff rubber material that provides a generally rigid mounting portion for selectively coupling support element 70, 80 to a patient interface assembly.

For example, a rubber material having a hardness of about 40 shA (Shore A) has been found to be suitable in one embodiment. In other embodiments, materials having a hardness from about 30 shA to about 80 shA have been used. Second portions 74 and 84 of support elements 70 and 80, respectively, are preferably formed from a generally less stiff rubber material than corresponding first portions 72 and 82 in order to provide for a comfortable engagement with the patient. For example, soft rubber materials having a hardness generally in the range of about 2 ShA to about 10 ShA or 20 to 80 Shore 00 have been found to be suitable. As shown in the example of FIG. 7, a further aperture 89 may be provided in first portion 84 to selectively adjust the properties support element 80, as will be discussed further below.

FIG. 8 shows an embodiment of a support element 90 that includes similar elements to support elements 50, 70 and 80 previously discussed. Like support element 80 of FIG. 7, support element 90 also includes an aperture 92 formed in a support face 94 and a second aperture 96 formed in first portion 98. The presence of one or more apertures in addition to aperture 92 provide an improved damping mechanism to support element 90. Aperture 92 is preferably generally circular in shape and has a diameter α. Second aperture 96 is likewise preferably circular in shape and has a diameter θ. Hollow cavity 100, is generally defined by a diameter β. In order to provide preferred damping properties, the diameter α of aperture 92 in support face 94 is preferably in the range of about 20% to about 40% of the diameter β of cavity 100. The diameter θ of aperture 96 in first portion 98 is preferably in the range of about 5% to about 15% of the diameter α of aperture 92 in support face 94.

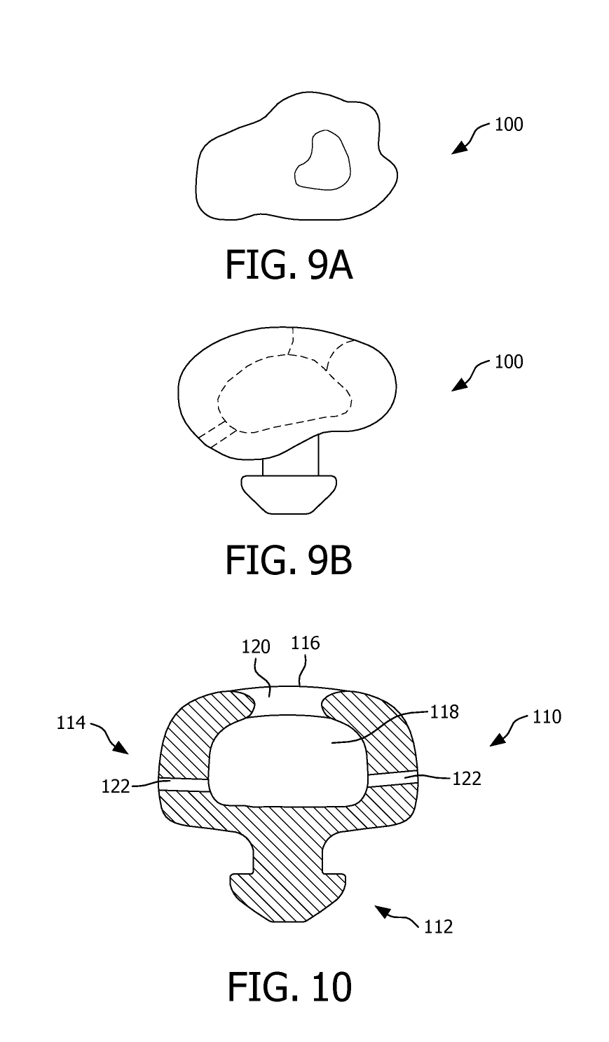

FIGS. 9A and 9B depict top and side views, respectively, of a support element 100 according to the principles of the present invention. Support element 100 includes similar elements as support elements 50, 60, 70 and 90 previously discussed. However, unlike those support elements previously discussed that were of a generally fixed geometrical shape, support element 100 demonstrates an example of the present invention having a generally irregular, free-form geometry while still providing similar functionality as that described for support elements 50, 60, 70 and 90.

FIG. 10 shows an embodiment of a support element 110 that, similar to support elements previously discussed, includes: a first portion 112 adapted to be coupled to a portion of the patient interface system; a second portion 114 having a support face 116 that is adapted to contact the patient; a cavity 118 defined therein; and an aperture 120. Unlike the support elements previously discussed, support element 110 further includes a number of apertures 122 (2 shown in FIG. 10) disposed generally in second portion 114 adjacent first portion 112. Such embodiment provides for an alternate arrangement for providing damping similar to as previously described.

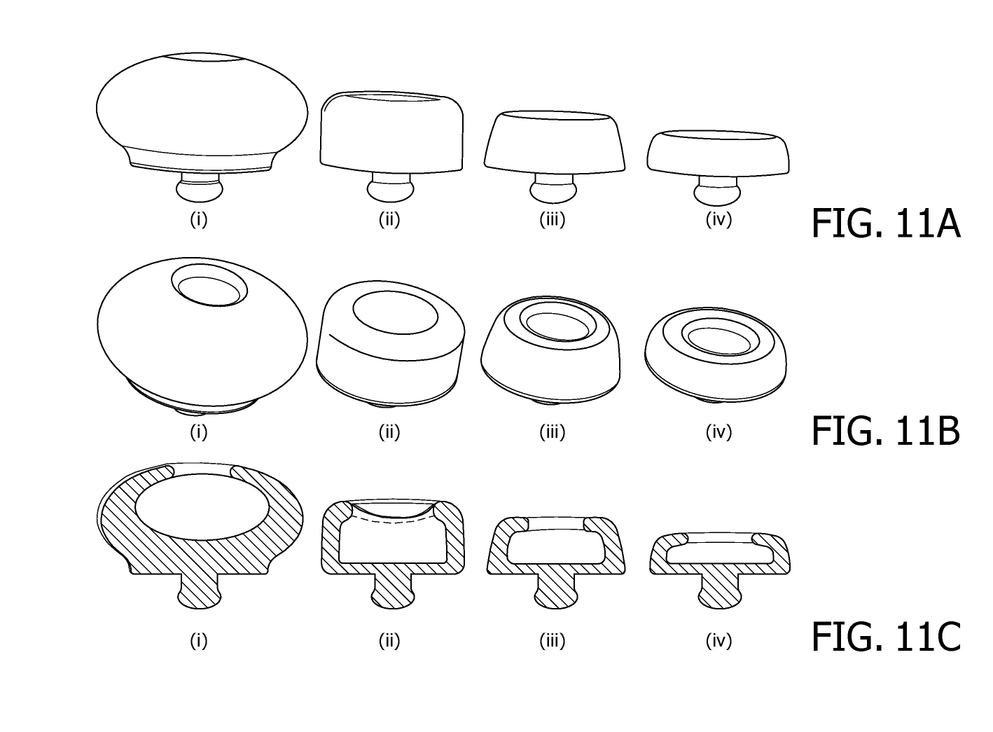

FIGS. 11A, 11B and 11C show elevation, isometric, and cross-sectional views, respectively, of support elements of varying size and geometry according to the principles of the present invention.

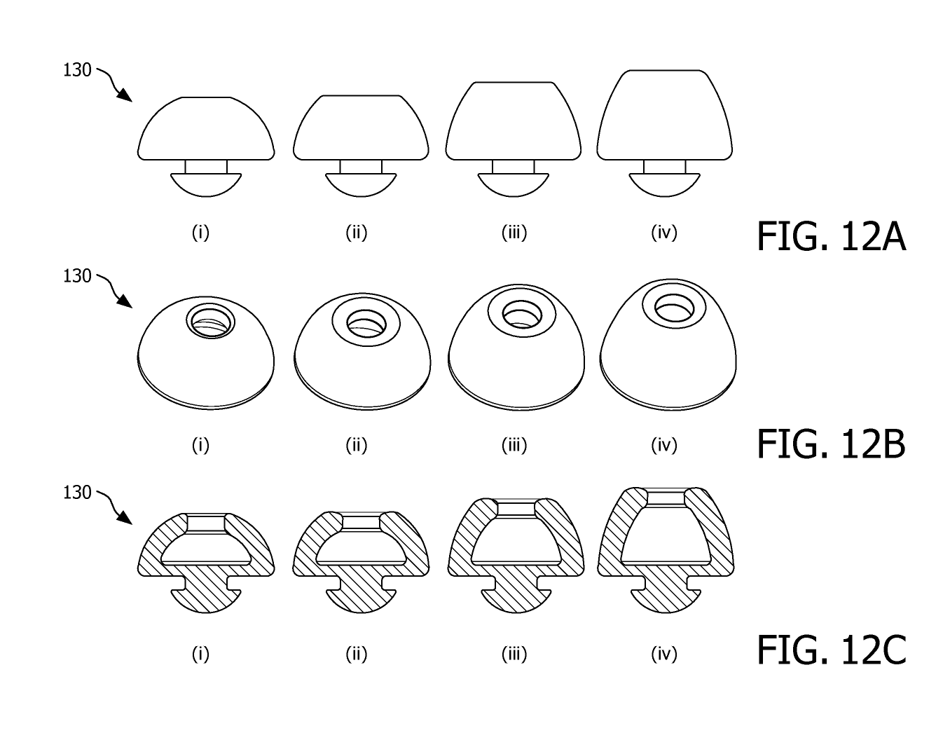

FIGS. 12A, 12B and 12C show elevation, isometric, and cross-sectional views, respectively, of an array 130 of support elements of varying size and similar geometry according to the principles of the present invention. Array 130 may be provided in a kit supplied to a provider or directly to a patient to provide for a custom-fit application. In such application, the provider or user can select a support element or elements of a desired size to best meet the needs of a particular application. Such array 130 is shown for example purposes only and is not intended to be limiting upon the present invention. It is to be appreciated that one or more characteristics of the support elements (e.g., without limitation, quantity, size, geometry) included within a kit may be varied without varying from the scope of the present invention.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A support element for use with a patient interface system, the support element comprising:
a first portion having a coupling mechanism adapted to be coupled to the patient interface system; and
a second portion having a support face adapted to contact the patient, the first and second portions defining a cavity therein, the support face having an aperture disposed therein providing access to the cavity wherein the aperture (60) in the support face is defined by a first diameter (α) and the cavity is defined by a second diameter (β) which is larger than the first diameter (α).

2. The support element of claim 1, wherein first portion and the second portion are portions of a unitary element.

3. The support element of claim 1, wherein the first portion is formed from a first material and the second portion is formed from a second material, the first material being stiffer than the second material.

4. The support element of claim 3, wherein the first material has a stiffness of about 40 ShA.

5. The support element of claim 3, wherein the second material has a stiffness in the range of about 2 ShA to about 10 ShA.

6. The support element of claim 1, wherein the first diameter (α) is in the range of about 20 percent to about 40 percent of the second Diameter (β).

7. The support element of claim 1, wherein one of the first and second portions comprises a second aperture disposed therein providing access to the cavity.

8. The support element of claim 7, wherein the second aperture is defined by a second diameter (θ) and the second diameter is in the range of about 5 percent to about 15 percent of the first diameter (α).

9. A system for providing a flow of gas to a patient, the system comprising:
a patient interface device;
a headgear assembly coupled to the patient interface device and adapted to secure the patient interface device to the head of the patient; and
a support element as recited in claim 1.

10. A kit for fitting a patient interface assembly to a patient, the kit comprising:
a plurality of support elements as recited in claim 1.

11. The kit of claim 10, wherein each support element of the plurality of support elements comprises a characteristic that varies from another of the plurality of support elements.

12. The kit of claim 11, wherein the characteristic is selected from the group consisting of: size and geometry.

13. The system of claim 9, further comprising a second support element as recited in claim 1.

14. The system of claim 9, wherein the patient interface device comprises a first end portion and an opposite second end portion, wherein the support element is disposed at the first end portion and wherein the second support element is disposed at the second end portion.
